(19) United States
(12) Patent Application Publication
(10) Pub. No.: US 2026/0015298 A1
(43) Pub. Date: Jan. 15, 2026

(54) PRODUCTION OF SYNGAS FROM METHANOL PRODUCED FROM SYNGAS AND/OR CO2

(71) Applicant: Olaf KÜHL, Greifswald (DE)

(72) Inventor: Olaf KÜHL, Greifswald (DE)

(21) Appl. No.: 18/995,212

(22) PCT Filed: Jun. 7, 2023
(86) PCT No.: PCT/EP2023/065221

§ 371 Date: Jan. 16, 2025

(30) Foreign Application Priority Data
Jun. 9, 2022 (DE) 10 2022 001 997.3

(51) Int. Cl. C07C 1/12; C01B 3/22; C01B 32/40; C10J 1/26

(52) U.S. Cl. CPC C07C 1/12; C01B 3/22; C01B 32/40; C10J 1/26; C01B 2203/0277; C01B 2203/061; C01B 2203/062; C01B 2203/1223; C10J 2300/1693

(57) ABSTRACT

The present invention relates to a process for the production of syngas by thermal catalytic decomposition of methanol produced from a mixture comprising at least a carbon oxide (CO and/or CO2) and hydrogen.

### Technical Problem

In power-to-liquid applications (PtL), carbon dioxide CO₂ is reduced to carbon monoxide CO using hydrogen H₂ as the reducing agent and a reverse water gas shift reaction (RWGS) in a shift reactor. The reaction is an equilibrium reaction that requires high temperatures and/or a catalyst. If the product syngas (n CO+m H₂), generated by adding additional hydrogen before or after RWGS, is then converted to alkanes, any inert gases (mainly CO₂) will be disadvantageous as these inert gases shorten the growing carbon chain causing increased production of methane, ethane, liquefied petroleum gas (LPG; propane and butane) and naphtha (pentane, hexane) at the expense of more valuable products like gasoline, kerosene, diesel as well as hard and soft waxes.

Since these inert gases are not consumed in the reaction, their relative content in the feedstock increases, making it more disadvantageous to proceed with converting the residue. As Fischer-Tropsch (FT) applications usually require three consecutive conversion cycles, this becomes a serious problem. The overall yield drops, a low value tail gas remains, and it becomes necessary to perform costly tail gas upgrading or recycling steps to increase the overall yield. A similar problem occurs in processes where a syngas containing inert gases is an intermediate in the production of synthetic fuels using a Fischer-Tropsch reactor as the syngas conversion step.

Such processes include, but are not limited to, pyrolysis of biomass, wet or dry reforming of renewable natural gas (RNG) (or natural gas (NG)) and the gasification of coal. Some of these processes use a shift reaction or the addition of external hydrogen to reach the preferred syngas composition in CO and hydrogen, thus increasing the CO₂ content further.

In the steel making industry, the DRI (direct reduction of iron oxides) process uses a syngas as reduction gas that is produced from the dry reforming of methane with CO₂ derived from the steel furnace's exhaust gas. The procurement of sufficient amounts of natural gas (or biogas) has become a difficult challenge recently and threatens the plans to convert from coal to renewable alternatives (CH₄, H₂).

The object underlying the present invention is to provide syngas (CO/H₂) preferably from biomass sources or electrolysis at ratios and compositions optimized for subsequent processes such as Fischer-Tropsch applications, reduction of iron oxides and synthesis of polyketones. Further considerations include the provision of said syngas with a better quality, higher purity, and a higher carbon and energy efficiency than prior art technologies. Especially for utilisation in Fischer-Tropsch applications, the absence of inert gases (CO₂, water, CH₄) is of vital importance for process performance, as is the almost complete absence of H₂ and CO₂ for polyketones.

### STATE OF THE ART

The Fischer-Tropsch (FT) process requires syngas of the composition CO+2 H₂ as feedstock. Traditionally, the syngas is provided by coal gasification or natural gas reformation. In a low carbon future, the feedstock will have to be biogenic or atmospheric CO₂ or biomass with the hydrogen produced by electrolysis of water.

The state-of-the-art in industrial syngas production is described in conclusive detail by Ke Liu, Chunshan Song, Velu Subramanian in: “Hydrogen and Syngas Production and Purification Technologies” published by the American Institute of Chemical Engineers in 2010. Starting from electrolysis, CO₂ and H₂ can be reacted in a RWGS-reactor (reverse water gas shift)

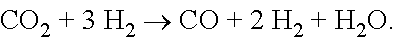

Other possible products are methane (by Sabatier-reaction or methanisation), methanol (DHC, direct hydrogenation of CO₂) or ethanol (CO₂ fermentation 2 CO₂+6 H₂→C₂H₅OH+3 H₂O). These primary products can then be reformed to syngas. The reformation of natural gas (methane, RNG) in a POx-reactor (POx: partial oxidation) is the standard technology in gas-to-liquids (GtL)-plants for synfuels, whereas the POx-reformation of methanol is known in the industrial production of acetic acid (WO 2006/005 269 A2). Ethanol can be steam reformed to produce a mixture of H₂, CO and CO₂ (Liu 2010). According to Liu 2010, steam reforming of methanol proceeds by oxidation of methanol to formic acid, dehydration of formic acid to formaldehyde and subsequent thermal decomposition of formaldehyde to CO and hydrogen

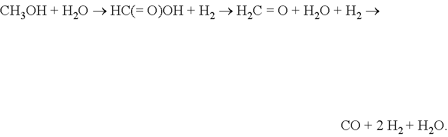

However, since the catalyst used for steam reforming of methanol is the same that is used for the WGS (water gas shift) reaction, the reaction sequence continues

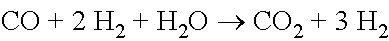

to form CO₂ and hydrogen as final products.

This is a common feature in syngas chemistry. A catalyst capable of enabling a certain desired reaction is also a catalyst for an undesired by-product. Examples include nickel (Catalysis Today, 1998, pp 191-6), which is a methanol decomposition catalyst at low temperatures (T=200° C.), but also enables methanation (CO+4 H₂→CH₄+2 H₂O) lowering the syngas yield significantly. Another example is ruthenium, which decomposes methanol at T=200° C. (Surface Science 1987, pp 832-41), but forms Ru(CO)₅, if reacted with methanol under high pressure.

This touches general selection parameters for experts in the art to select chemical reactions for technology pathways favoured in industrial processes. Small reactors with low capex costs require high pressure, if gases are the feedstock and/or the products. An increase in pressure disfavours decomposition of methanol, as the number of gas molecules is increased from one to three in methanol decomposition. This in turn causes an increase in reaction temperature to counteract the pressure increase, which might damage the catalyst and certainly compromises energy efficiency.

The expert in the field knows that a reaction suitable for a small-scale laboratory experiment may not necessarily be suitable for an industrial production line capable of producing up to 40 t/h in a single reactor. It is therefore not surprising that despite a multitude of research publications for the decomposition of methanol at T=200° C. prior to 2010, Liu 2010 mentions only one industrial decomposition pathway for methanol that decomposes CH₃OH neatly into CO+2 H₂. This technology is plasma reforming. A reactor designed for the plasma reforming of methane is described in US 2014/0364516 A1 (awarded to Kühl). The apparatus is also capable of decomposing methanol and in fact does so to produce the plasma gas (which is syngas) from methanol during start-up. However, the energy consumption is twice that of a catalytic methanol decomposition technology and the plasma route has far higher capex costs and is burdened with severe safety challenges (the plasma chamber operates near the limits of known reactor materials and beyond the temperature limits of most safety features).

Thus, the expert in the art knows the standard POx-reforming of methane to produce syngas for the FT-process. However, this route, although applied in Trinidad, is restricted to the availability of RNG. The expert also knows of the RWGS-reactor that is limited to the availability of renewable electricity (mainly wind and solar) on site (as the hydrogen has to be produced by electrolysis of water). The expert also knows of the POx-reformation of methanol that loses about 30% of the methanol to oxidation to CO₂ and water and of plasma reforming that has severe cost, efficiency and safety issues.

The expert would appreciate a methanol decomposition reactor such as is mentioned in DE 20 2011105262 U1. DE 202011105262 U1 reports that a catalyst capable of neatly splitting methanol to CO and hydrogen at T=200° C. does exist, but fails to disclose the identity or the reaction parameters of said catalyst. It therefore fails to disclose the parameters necessary for the expert in the field to decide on its suitability for an industrial application or in fact to use this process.

### Technical Solution

According to the present invention, a process for the production of syngas by thermal catalytic decomposition of methanol produced from a mixture comprising at least a carbon oxide (CO and/or CO₂) and hydrogen is provided.

More specifically, a process for the production of syngas is provided, which comprises the steps of:
(i) Producing methanol from a syngas composition (a),
(ii) Optional separation of the methanol,
(iii) Optional drying of the methanol,
(iv) Optional purification of the methanol,
(v) Optional storage of the methanol,
(vi) Optional transport of the methanol,
(vii) Producing syngas (b) from methanol obtained in step (i) by thermal catalytic decomposition,
(viii) Optionally subjecting the syngas (b) to a separation step in a separation unit, wherein CO enriched and H₂ enriched streams are obtained,
(ix) Optionally subjecting the syngas (b) or any CO enriched and/or H₂ enriched streams obtained therefrom to a Fischer Tropsch (FT) synthesis step.
(x) Optional transfer of the syngas (b) or any CO enriched and/or H₂ enriched streams obtained therefrom to a DRI unit for iron oxide reduction,
(xi) Optional transfer of the syngas (b) or any CO enriched and/or H₂ enriched streams obtained therefrom to a CO polymerisation unit.

The present invention proposes, in particular, to generate methanol from syngas and/or CO₂ as an intermediate, which methanol, optionally after separation, storage and/or transport is thermal catalytically decomposed into a syngas comprising CO and H₂ at optimized ratios, which can be then directly used, in particular, in Fischer Tropsch synthesis and for the reduction of iron oxides.

Furthermore, the present invention allows to purchase the methanol produced in step (i) from a third party and using either none, any or all of the methods in steps (ii) to (vi) to produce syngas (b) from it in step (vii).

It is step (vii) that is crucial in the sequence of the present invention, the expert uses two equations

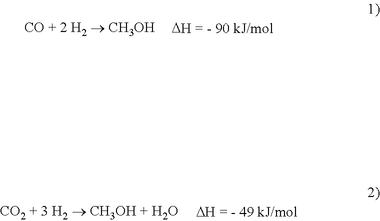

and a family of commercially available catalysts based on Cu/ZnO/Al₂O₃ to synthesise methanol. It is known to the expert that a thermodynamic equilibrium reaction yielding a product can be reversed to produce the starting material, if the reaction conditions are changed. In the present case, the expert can use two obvious factors. Increasing the reaction temperature in an exothermic reaction causes decomposition of the product and thus formation of the starting material. Likewise, decreasing the pressure in a reaction that proceeds with an increase of gaseous molecules, as methanol decomposition does, is favourable for this reaction. Therefore, the expert would use either of the two methanol synthesis reactions, lower the reaction pressure and increase the temperature beyond the reaction temperature of methanol synthesis. These are usually p=40 bar and T=240° C. to 270° C.

The expert is now faced with two problems: It is known from methanol synthesis that the catalyst is sensitive to high temperatures. At temperatures above T=300° C., the Cu component degrades rapidly, the catalyst loses performance and has to be replaced in unacceptably short intervals. The expert would therefore not wish to increase the reaction temperature beyond T=300° C.

The other problem is that the expert knows that methanol synthesis from syngas only proceeds, if at least 1-3% CO₂ is added. As the presence of CO₂ is seemingly crucial to methanol formation, any reversal of this reaction, according to the principles of thermodynamics, will have to yield some CO₂, a compound that is undesired in the intended syngas. Therefore, the expert is interested in the methanol decomposition in the presence of water. This reaction CH₃OH+H₂O→CO₂+3 H₂

is known as methanol-steam-reforming MSR and a major route in the industrial production of hydrogen. Liu 2010 explains the generally accepted mechanism of this reaction in great detail. According to the experts, there are two possible mechanisms

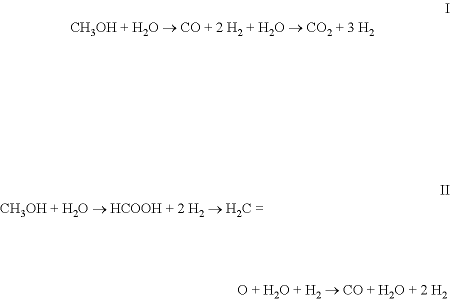

The presence of CO is detected spectroscopically, but at low amounts, too low to support pathway I. The kinetic simulations also rule out pathway I. The experts therefore conclude that methanol steam reforming proceeds exclusively via pathway II. This explicitly rules out, in the eyes of the expert, that methanol spontaneously decomposes to CO and hydrogen (pathway I) in parallel with pathway II. This view is supported by the mechanism of CO₂ hydrogenation to methanol (reaction 2) above).

The mechanism of the methanol synthesis reaction from CO₂ is published in Chem. Soc. Rev. 49 (2020), pp. 1385-1413 and shown in FIGS. 6 and 7. This mechanism features all the principle intermediate compounds of pathway II coordinated to the surface of the catalyst. Decomposition of methanol to CO would proceed via the same methoxy and methenoxy (H₂CO) intermediates. Decomposition to CO₂ would then follow the route via the dioxygenyl-complex, while the route to CO needs to form the formyl-complex directly. However, the expert knows that dry syngas does not form methanol and that therefore, the route from the methenoxy-to the formyl-complex is not thermodynamically viable. The expert also knows that this pathway I is not observed in methanol-steam-reforming. The expert does know that formaldehyde, the compound formed by the dissociation of the methenoxy-species from the catalyst surface, decomposes at temperatures above 570° C. to CO and hydrogen (Fletcher, Proc. Soc. Roy A, 42, pp 357), a temperature that would damage the catalyst. What the expert seemingly does not know is that addition of water, instead of CO₂, to the syngas in methanol synthesis enables the formyl-complex to form the formiate-complex in the mechanism of methanol synthesis from CO₂. The expert of course knows that the same catalyst system catalyses methanol synthesis and the WGS-reaction (CO+H₂O→CO₂+H₂) and that water deactivates the catalyst and therefore the expert removes the water from the syngas.

The present invention is based on the following observation. In methanol synthesis from syngas, initially the formyl-complex is formed. The formyl-complex is unstable and rapidly decomposes back to CO and hydrogen. Formation of the methenoxy-complex from the formyl-complex would be the rate determining step in methanol synthesis from CO, but the formyl-complex is too unstable for the methenoxy-complex to be formed. If water is added to the reaction, formation of the formiate-complex becomes the rate-determining step as it is faster than formation of the methenoxy-complex. It is also fast enough to occur within the lifetime of the formyl-complex. Therefore, addition of water brings the hydrogenation of CO onto the same reaction pathway as is observed in the hydrogenation of CO₂. If CO₂ is added to the reaction, the CO₂ is hydrogenated to methanol and forms water as a by-product. The water can then oxidise the formyl-complex of the CO-pathway to the formiate-complex. The water is released towards the end of the cycle (upon formation of the methenoxy-complex).

In the decomposition of methanol, the methenoxy-complex is formed and has three options: a) dissociation to formaldehyde, b) formation of the dioxygenyl-complex (en route to formiate) by addition of water and c) dehydrogenation to the formyl-complex (route first disclosed by the present invention). Increasing the amount of water would therefore favour CO₂ formation, while the absence of water favours CO formation. The formiate-complex can eliminate H₂ to form CO₂ or water to form CO. The more water is present, the less likely water elimination from the formiate-complex becomes. As a result, more CO₂ is formed and more water is consumed.

The present invention discloses the viability of the hydrogenation of the formyl-complex to the methenoxy-complex in the generally accepted mechanism of CO₂ hydrogenation. It also discloses the possibility to oxidise the formyl-complex to the formiate-complex by water. In doing so, the present invention fully explains the hitherto partly unknown mechanism of methanol synthesis from syngas and incorporates it into the mechanism of CO₂ hydrogenation. The present invention discloses a new pathway and process for the low temperature decomposition of methanol to syngas.

In the absence of water, the dehydrogenation of the methenoxy-complex to the formyl-complex is the most favourable option. The oxidation of the methenoxy-complex to the dioxygenyl-complex requires water. As the formyl-complex is unstable and hydrogenation to the methenoxy-complex very unfavourable, irreversible decomposition to CO and hydrogen is the only option. Formation of formaldehyde will not be observed as dissociation from the catalyst surface is reversible and irreversible decomposition via the formyl-complex removes the formaldehyde precursor from the equilibrium. Addition of water provides two options: i) the dioxygenyl-complex can be formed and ii) the formyl-complex can be oxidised to the formiate-complex by the water. Both options yield CO₂, which is observed. The CO/CO₂ ratio in the final product becomes a function of the water content and is independent of the initial decomposition step.

In the absence of the formyl ↔ methenoxy pathway disclosed by the present invention, the expert in the field has to expect a catalytic dehydrogenation of methanol followed by formaldehyde dissociation. The formaldehyde then thermally decomposes above T=570° C. to CO and hydrogen. At this temperature, the expert assumes catalyst sintering and deactivation.

It is therefore more promising for the expert to develop a hydrogenation catalyst capable of dehydrogenating both, methanol and formaldehyde.

The addition of water to a syngas used to produce methanol is not obvious to the expert, as the expert knows that the Cu/ZnO/Al₂O₃ catalyst system is an excellent WGS-catalyst that converts CO and water to CO₂ and hydrogen, the benchmark technology for industrial hydrogen production from syngas generated by natural gas reformation.

Alternatively, the methanol can be decomposed in a shaft furnace, as is utilised in the direct reduction of iron oxides (DRI). The methanol is fed into the shaft furnace as a hot methanol vapour instead of the hot syngas used in the state-of-the-art DRI process.

The hot methanol vapour is thermal-catalytically decomposed by the iron oxide (Fe₂O₃, Fe₃O₄ or other iron oxide containing ores) to a syngas which then in turn reduces the iron oxide to iron (HBI; hot briquetted iron) according to the state-of-the-art DRI process. The iron oxide causes the dehydrogenation of methanol to formaldehyde, which then thermally decomposes to CO and hydrogen

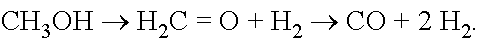

The thermal decomposition of formaldehyde occurs, even without catalyst, at temperatures above T=570° C. and thus below the temperature of T=700° C. required for a DRI process run with pure hydrogen.

Iron oxides can either be reduced by CO or by hydrogen or by any mixture of the two (syngas). It is well known that the reduction of iron oxides (hematite Fe₂O₃, magnetite Fe₃O₄) with CO is exotherm and that with hydrogen is endotherm, but kinetically faster. A further complication is the oxidation of iron to iron oxides by steam (liberation of hydrogen)

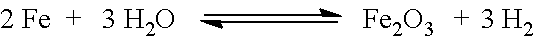

That does not occur with CO₂. Therefore, the reduction with hydrogen needs a reaction temperature of about T=700° C. to prevent the re-oxidation of iron to iron oxides by steam, whereas it is known from the blast furnace process that reduction with CO occurs at temperatures even below T=400° C., albeit slowly and without re-oxidation.

The present invention provides syngas, CO and/or hydrogen that fulfils any international criteria of biogenic and renewable and is thus CO₂ neutral at any location required by the steel manufacturer, since methanol can easily be stored and transported. It can be used in the manufacture of steel that is known as green steel.

Therefore, the present invention provides the steel manufacturer with the choice to a) decompose the methanol to CO and hydrogen, optionally remove part of the hydrogen and reduce the iron oxide at low temperature or optionally use part of the hydrogen for other purposes or b) use neat hot methanol to reduce the iron oxide at high temperatures.

### Technical Effect

It might seem to be of little practical use to produce methanol for the sole purpose of decomposing it back into its original components and amounts. Depending on the carbon oxide composition used in step (i), the reaction sequence for example includes the following chemical equations:

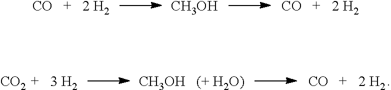

However, this reaction sequence (including the step of methanol production (i)—and methanol decomposition (vii)) is capable to eliminate all impurities (especially CO₂) from e.g. a shift reactor product stream and sets the H₂: CO ratio of a subsequent FT-feedstock to e.g. preferably about 2:1, a ratio that is suitable for the conversion to alkanes.

The H₂: CO ratio can easily be manipulated to all other preferred ratios by use of a separation unit, for example by utilising a membrane filtering unit that with the aid of a pressure drop across a membrane, allows the fast-moving gas (here hydrogen) to pass through the membrane while the slow-moving gas (here CO) does not pass through the membrane and leaves the unit through another exit. It is also capable to deliver the optimised syngas stream continuously and independent of a volatile electricity production, thus eliminating the necessity of flexible FT-reactors, H₂-storage tanks or electricity storage devices.

Methanol synthesis (a C₁ compound) and C—C bond forming reactions like FT-conversions respond differently towards the presence or absence of inert gases (like CO₂, nitrogen or methane). During the C—C bond forming step in the FT-process, two C-atoms need to occupy adjacent active sites on the catalyst. If the active site adjacent to the growing C-chain is occupied by an inert gas molecule, chain termination is the likely result. Thus, in FT-reactions, the presence of inert gases results in shorter chain lengths and a higher proportion of methane, ethane and other low-value short-chain products as well as a generally lower yield compared to the absence of inert gases. In methanol synthesis, no C—C bond formation occurs. That limits the product range essentially to methanol. The influence of inert gases is reduced to a general yield reduction that can be prevented by a corresponding increase of the reactor size.

According to the process of the present invention it is possible to provide a syngas composition with a desired fixed H₂: CO ratio in particular in the “stochiometric” ratio of about 2:1 and the absence of inert gases like CO₂. As mentioned before, it is further possible by dividing the product stream into CO enriched or H₂ enriched streams in a separating unit to provide any desired ratio such as for example a CO/H₂ ratio of about 1:1 to 1:4, preferably 1:1.5 to 1:3, more preferably 1:1.6 to 1:2, most preferably of about 1:1.7 to 1:1.8. Likewise, in a cascade of FT reactors the CO depleted streams can be supplemented again by any H₂ enriched stream from the methanol decomposing unit used in step (vii).

Furthermore, the changed optimized syngas composition results in a large shift of the FT-product distribution towards hard and soft waxes and middle distillate and the reduction of low-value products like methane, ethane and LPG.

The capital expense of the methanol loop is at least partially offset by the redundancy of the main RWGS (or shift) reactor, the additional shift reactors and steam methane reforming (SMR) for the recycling of FT tail gases in the state-of-the-art pathway. The present invention also decreases energy consumption and operating costs.

A further beneficial technical effect results from the volatility of most renewable electricity sources, especially wind and solar power. Converting syngas from the shift reactor or CO₂ and hydrogen directly into methanol enables the storage and transport of this intermediate product. Conversion of the methanol to alkanes can then be achieved by reactors of smaller capacity as they can then be operated with a much higher load factor. It also makes the flexibilization of the FT-reactor obsolete. This is a major advantage, as there is presently no known solution to enable FT-reactors to adjust its production according to changing electricity availability (e.g. wind and solar power).

The present invention enables the storage and transportation of syngas to another location in the form of methanol. The conversion of the syngas to alkanes can then be performed at this remote location and in a continuous operation. It then becomes possible to use gaseous by-products of the FT-process (hydrogen, CO, methane, ethane, LPG) at this other location, where they may be of greater use or have a higher value.

For example, carbon dioxide can be reacted with hydrogen obtained by electrolysis of water with electricity from renewable electricity sources, especially wind and solar power, and the resulting methanol can be stored, transported and retransformed into a syngas of a desired composition for further use at any other location globally.

It also means that locations with limited carbon feedstock abundance (CO₂, biomass, etc.) can be used to produce methanol, the methanol can easily be transported to a central production site, where it is converted to the final products (FT etc.) with greater efficiency, with greater economy-of-scale and more options to utilise otherwise stranded by-products.

Another preferred syngas composition is almost pure CO for use in a DRI process of steel manufacture. The beneficial technical effects include a lower reaction temperature with accompanying higher energy efficiency, the use of the hydrogen for other purposes (hydrogen carrier) and the possibility of steel production with negative carbon emissions, if the steel furnace is connected with a CCS unit (CCS: carbon capture to storage) or the CO₂ is transported back to the methanol production site (circular economy).

### DETAILED DESCRIPTION OF THE INVENTION

The present invention is directed at a process for the production of syngas by thermal catalytic decomposition of methanol produced from a mixture comprising at least a carbon oxide (CO and/or CO₂) and hydrogen, i.e., from syngas. It is noted that the technical definition of syngas usually refers to a mixture of hydrogen and carbon monoxide in various ratios, wherein the gas may contain some carbon dioxide and/or methane. According to the invention, the gas composition referred to as syngas, which is used for the production of the methanol which is subjected to thermal catalytic decomposition in the process, may also comprise CO₂ and hydrogen in the absence of CO. This composition, which preferably, but not mandatorily, comprises CO is referred to as “syngas composition (a)” in the following.

In contrast, the syngas composition obtained by the decomposition of methanol in the process necessarily comprises CO and H₂, and preferably CO is the major carbon-containing component of the gas composition. The syngas composition obtained from the decomposition of methanol in the process of the invention is referred to as syngas or “syngas (b)” in the following.

Preferably, the process for the production of syngas comprises the steps of:
(i) Producing methanol from a syngas composition (a),
(ii) Optional separation of the methanol,
(iii) Optional drying of the methanol,
(iv) Optional purification of the methanol,
(v) Optional storage of the methanol,
(vi) Optional transport of the methanol,
(vii) Producing syngas (b) from methanol (methanol decomposition step) obtained in step (i) by thermal-catalytical decomposition,
(viii) Optionally subjecting the syngas (b) to a separation step in a separation unit, wherein CO enriched and H₂ enriched streams are obtained,
(ix) Optionally subjecting the syngas (b) or any CO and/or H₂ enriched streams obtained therefrom to a Fischer Tropsch (FT) synthesis step
(x) Optionally using the syngas (b) or any CO and/or H₂ enriched streams obtained therefrom for the reduction of any iron oxide to iron, wherein optionally the process is performed by two or more individual parties.

The syngas composition (a) used in step (i) comprises a carbon oxide, selected from CO and/or CO₂, and hydrogen. Preferably, it comprises CO and H₂, and optionally also CO₂. A stream of syngas composition (a) that is typically, but not necessarily, the tail gas of a shift reactor, is dried by condensing the water in a condenser and separating the gas phase from the liquid phase. Preferably, the composition of the syngas is adjusted thus that it contains 1% to 10%, more preferred 4% to 6% by weight of a mixture of CO₂ and water related to the overall weight of the syngas composition.

Further preferably, the syngas composition (a) used in step (i) has a molar ratio of carbon oxide to hydrogen in the range of 1:4 to 1:1 more preferably 1:3 to 1:2.

It is also preferred that in the process for the production of syngas of the invention, the carbon oxide in the syngas composition (a) used in step (i) comprises carbon dioxide (CO₂). The syngas then enters the first methanol synthesis reactor and is converted to methanol (step (i)). The methanol is separated from the gaseous compounds (in particular CO₂, other inert gases, CO, H₂, CH₄) and water.

In a preferred embodiment of the process for the production of syngas, the syngas composition (a) used in step (i) is obtained from subjecting carbon dioxide and hydrogen to a reverse water gas shift reaction (CO₂+H₂ [symbol] CO+H₂O).

In the process of the invention, the methanol from the methanol synthesis reactor is sent to the methanol decomposition reactor (step (vii)).

According to the invention, each if the terms “methanol decomposition”, “methanol decomposer” and “methanol decomposition reactor” refer to a reactor in which methanol is thermal-catalytically decomposed, preferably using a catalyst as described herein as being preferred according to the present invention. It is noted that the term “reactor”, which refers to a space in which a reaction takes place, may also refer to reactors different from reactors designated for methanol decomposition or producing methanol as described herein, but also to other types of reactors, in particular to a furnace in which iron oxides are reduced to iron.

It is preferred that dried methanol is used in the methanol decomposition step (vii), preferably methanol comprising less than 10 wt.-% water, more preferably less than 1 wt.-% water, even more preferably less than 0.5 wt.-% water, most preferably less than 0.2 wt.-% water, is used in the methanol decomposition step (vii) and is fed into the methanol decomposition unit, and wherein the reaction is carried out at a temperature below 300°. In the methanol decomposition reactor, the methanol is thermally decomposed (with the use of catalysts) to form syngas (b). In a preferred embodiment according to the invention, step (vii) is carried out with one or more of the following conditions:
(a) a temperature in the range of 100° C. to 400° C., preferably of 200° C. to 350° C., more preferably of 220° C. to 300° C. and most preferably of 240° C. to 270° C.
(b) a pressure in the range of 1 bar to 70 bar, preferably 20 bar to 50 bar, more preferably 35 bar to 45 bar,
(c) the use of dried methanol comprising less than 10 wt.-% water, preferably less than 1 wt.-% water, more preferably less than 0.5 wt.-% water, most preferably less than 0.2 wt.-% water,
(d) a catalyst selected from the group consisting of compounds of the elements Rh, Pd, Pt, Ir, Ru, Fe, Zn, Co, Ni, Cu and Mn, preferably from the elements Zn, Cu, Fe, Pt, Pd and Rh, more preferably from the elements Zn, Cu and Fe and most preferably catalysts containing either ZnO, a combination of ZnO and Cu or iron oxides, in particular Cu/ZnO, Cu/ZnO/Al₂O₃, doped Cu/ZnO, doped Cu/Zn/Al₂O₃, Fe₂O₃ or Fe₃O₄.

According to the stoichiometry of methanol decomposition, the syngas (b) is expected to be of the composition essentially: CO+2 H₂.

In a preferred embodiment according to the invention, in step (i) and step (vii) the same reactor type is used, differentiated by the presence of a cooling device for step (i) and a heating device for step (vii), respectively.

In another preferred embodiment according to the invention, step (i) and step (vii) are operated with the same reactor type with the following differences:
(a) a methanol evaporating, compressing and heating unit,
(b) a heating device for the main temperature regulating cycle,
(c) reversal of the post-reactor recyclization unit so that the gaseous phase (syngas) is sent downstream and the liquid stream (methanol) is recycled back into the reactor.

The retransformation of the methanol obtained from the syngas composition (a) into the syngas (b) is carried out in a methanol decomposer. The methanol decomposer can be a standard syngas-to-methanol reactor that is retrofitted with the following modifications: the steam cooling cycle is equipped with a heating device outside the reactor, the recyclization unit is changed so that the gas phase (syngas) is now sent downstream and the liquid phase (methanol) is recycled back into the reactor. Further, the reactor needs to be equipped with or attached to a methanol evaporating, compressing and heating unit.

Alternatively, the methanol decomposer can be a standard methanol-steam-reformer that is operated with a sub-stoichiometric amount of water, preferably none at all. It should be mentioned that the CO₂ content in the product gas from such a methanol decomposer is directly equivalent to the amount of water fed into it. For applications in FT-processes, the methanol-steam-reformer type methanol decomposer should be operated without water.

In a preferred embodiment according to the invention, the process is performed in a methanol decomposer with dry and CO₂ free methanol, wherein a hydrogen enriched stream, preferably comprising at least 90 mol-% of H₂, is separated from the syngas stream obtained from step (vii), and wherein the remaining CO enriched stream, preferably comprising at least 10 mol-% of CO, is fed into a shaft furnace for the reduction of iron oxides to iron of step (x).

In another preferred embodiment according to the invention, in the process of the invention 1 wt.-% to 90 wt.-% water based on the amount by weight of methanol, preferably 10 wt.-% to 85 wt.-%, more preferably 20 wt.-% to 70 wt.-%, most preferably 20 wt.-% to 50 wt.-% water in addition to methanol is added in step (vii).

In a further preferred embodiment according to the invention,
the catalyst used in the thermal catalytic decomposition of methanol in step (vii) is a ZnO-containing catalyst, preferably containing ZnO/Al₂O₃, more preferably containing Cu/ZnO/Al₂O₃, and
an amount of water is added to the methanol subjected to decomposition in step (vii), preferably 1 wt.-% to 90 wt.-% water based on the amount by weight of methanol, preferably 10 wt.-% to 85 wt.-%, more preferably 20 wt.-% to 70 wt.-%, most preferably 20 wt.-% to 50 wt.-% water in addition to methanol is added.

In another embodiment of the invention, wherein step (vii) is performed in a shaft furnace upon vapourizing methanol and feeding it hot into the shaft furnace, followed by the reduction of iron oxides to iron of step (x). According to this embodiment, step (vii) and step (x) are preferably performed at temperatures between 500° C. and 950° C., more preferably between 550° C. and 850° C., still more preferably between 550° C. and 700° C. and most preferably between 550° C. and 650° C.

According to the invention, it is preferable that the syngas (b) obtained in step (vii) comprises carbon monoxide and hydrogen, in a molar ratio of CO to H₂ of about 1:1 to about 1:3, preferably of about 1:1 to 1:2.

The syngas stream can be used either directly or is fed into a separation unit. The separation unit in such step (viii) divides the syngas stream into a CO rich stream and a hydrogen rich stream.

One way to achieve this separation is to utilise a membrane filtering unit that with the aid of a pressure drop across a membrane, lets the fast-moving gas (here hydrogen) pass through the membrane while the slow-moving gas (here CO) does not pass through the membrane and leaves the unit through another exit. After the separation unit, the two gas streams can be re-mixed in a way so that a stream having a desired molar ratio CO/H₂ of about 1:1 to 1:4, preferably 1:1.5 to 1:3, more preferably 1:1.6 to 1:2, most preferably of about 1:1.7 to 1:1.8 results.

In a preferred embodiment according to the invention, the syngas (b) obtained in step (vii) is subjected to a separation step in a separation unit, wherein CO enriched or H₂ enriched streams are obtained, which optionally can be used directly in subsequent processes or after recombination with the main stream from the methanol decomposition unit used in step (vii), in particular, to adjust a certain CO/H₂ molar ratio. Further preferably, the CO enriched or H₂ enriched streams obtained are combined with a stream selected from:
the syngas stream resulting from the methanol decomposing unit used in step (vii) to provide CO enriched or H₂ enriched streams of a certain molar ratio, and
any stream leaving a FT reactor in the FT synthesis step (ix), to provide a combined CO/H₂ stream preferably having a molar ratio CO/H₂ of about 1:1 to 1:4, preferably 1:1.5 to 1:3, more preferably 1:1.6 to 1:2, most preferably of about 1:1.7 to 1:1.8. According to the invention, the syngas (b) or any CO enriched and/or H₂ enriched streams obtained therefrom are optionally subjected to a Fischer Tropsch (FT) synthesis step. In such FT synthesis step, the syngas of a desired composition is then preferably fed into a FT synthesis unit consisting of one or more, preferably two or more, more preferably 2 to3 FT-reactors (step (ix)).

In a preferred embodiment according to the invention, the syngas (b) obtained in step (vii) is transferred into a FT synthesis unit, wherein the syngas (b) is subjected to a FT synthesis step (ix), providing higher molecular products having two or more, preferably three or more carbon atoms selected from the group consisting of alkanes, alkenes or alcohols.

In another preferred embodiment according to the invention, the syngas (b) obtained in step (vii) is transferred to a FT synthesis unit, consisting of one or more, preferably two or more FT reactors and wherein a CO enriched stream leaving a FT reactor is recombined with a H₂ enriched stream preferably resulting from the separation step (viii), so as to preferably provide a stream having a molar ratio CO/H₂ of about 1:1 to 1:4, preferably 1:1.5 to 1:3, more preferably 1:1.6 to 1:2, most preferably of about 1:1.7 to 1:1.8.

In general, according to the invention the flue gas from the first FT-reactor is very CO enriched and can be mixed with enough of the hydrogen rich gas stream from the separation unit to adjust again the preferred composition (e.g. CO+1.7 H₂) before it enters the second FT-reactor. The procedure can be repeated for the third and any subsequent FT-reactor, if required.

It is known in the art that a syngas stream of a composition CO+2 H₂ (i.e. about 1:2) can be used directly in a FT reaction. Partial separation of hydrogen (as described) is not necessary for the present invention, but can improve the performance in many applications, such as steel manufacturing and CO polymerisation to polyketones.

In a preferred embodiment according to the invention, the syngas (b) obtained in step (vii) is subjected to a separation step in a separation unit, wherein a stream of CO is obtained that is used to polymerise CO to a polyketone.

Preferably, the purity of the CO in said stream is 85% v/v or more, more preferably 90% v/v or more, even more preferably 95% v/v or more, further preferably 98% v/v or more, still more preferably 99% v/v or more, still further preferably 99.5% v/v or more, and most preferably 99.9% v/v or more.

In another preferred embodiment according to the invention, the syngas (b) obtained in step (vii) is used to reduce an iron oxide to iron. Further preferably, the syngas (b) obtained in step (vii) is subjected to a separation step in a separation unit, wherein CO enriched or H₂ enriched streams are obtained, whereby either the one or the other is used for the reduction of iron oxide to iron, and wherein optionally
(a)—the H₂ content of the CO rich stream used for iron oxide reduction is modified according to an external hydrogen demand, and/or
(b) the H₂ rich stream is used to export hydrogen into a grid and/or a storage tank, and/or
(c) the H₂ rich stream is used for electricity generation, and/or
(d) the H₂ rich stream is used to convert part of the CO₂ from iron oxide reduction to produce methanol.

The process heat from the methanol synthesis reactor (90.4 KJ/mol) can be transferred to the methanol decomposition reactor using a heat exchanger. This at least partially offsets the enthalpy of decomposition (90.4 KJ/mol) required by the methanol decomposition reactor used in step (vii).

In a preferred embodiment according to the invention, at least part of the process heat of the methanol producing step (i) and/or of the FT synthesis step (ix) are transferred to the methanol decomposition step (vii).

The total yield from the cascade of at least one FT-reactor is almost 100% (including gaseous hydrocarbons). The tail gas of the last FT-reactor in the cascade can then be combusted to balance the methanol decomposition process heat and/or to contribute to the reaction enthalpy of the shift reactor. Alternatively, the energy of this tail gas can be converted to electricity to use in electrolysis (hydrogen synthesis).

In a preferred embodiment according to the invention, step (ix) is carried out and the tail gas of the FT-reactor(s) is combusted to provide heat for the methanol decomposition step (vii), and optionally the CO₂ from the combustion gas is recycled into the methanol synthesis step (i), wherein the tail gas preferably comprises CO₂, H₂ and CO.

The tail gas of the last FT-reactor can also be filtered with a membrane to recover the hydrogen (which is sent back to the shift reactor or the FT-reactors) and then treated as described above.

In a preferred embodiment according to the invention, the step (ix) is carried out and the tail gas of the FT-reactor(s) is recycled back into step (i), wherein the tail gas preferably comprises CO₂, H₂ and CO. Alternatively, CO₂ can be the only carbon containing starting material for the production of methanol in step (i). In this case, the shift reactor is omitted from the flow chart and the tail gas (CO₂, H₂ and small amounts of CO) is recycled back into the methanol synthesis reactor as feedstock.

More alternatively, if the starting material is a syngas that has an insufficient amount of hydrogen (as is the case in the pyrolysis of biomass), additional hydrogen can be added instead of producing the hydrogen internally by reacting part of the CO with water in a shift reactor (CO+H₂O [symbol] CO₂+H₂). In this case, the shift reactor, but not the methanol synthesis reactor, is omitted from the flow chart.

In a preferred embodiment according to the invention, the syngas (b) obtained in step (vii) is used for the production of hydrogen, ammonia, synthetic hydrocarbons for use as a fuel or lubricant, in particular via the Fischer-Tropsch process.

The syngas from the methanol decomposer can be used not only in a FT-reactor, but in any other syngas conversion process, such as in the production of steel, hydrogen, ammonia, synthetic hydrocarbons for use as a fuel or lubricant, in particular via the Fischer-Tropsch process. Such a process can be the production of hydrogen from methanol. The skilled person knows several such processes for the synthesis of hydrogen from methanol. The two standard processes are firstly steam reformation of methanol to hydrogen and CO₂ and secondly the reaction of methanol with water and oxygen (ATR process; autothermal reforming). Separation of hydrogen can then be realised by membrane, pressure swing adsorption (PSA) or carbon capture.

The syngas produced by decomposing methanol can be divided into essentially two separate streams of gas, the one containing mainly hydrogen, the other containing mainly CO. The two streams can then be used for separate purposes, respectively.

Similarly, the methanol synthesis reactor used in step (i) cannot only convert the syngas from a RWGS (reverse water gas shift reaction) reactor, but any other inert gas containing stream of syngas. Such a syngas can be the tail gas of a WGS-reactor (WGS: water gas shift) that reacts CO with water to yield hydrogen and CO₂. Pyrolysis of biomass typically yields a syngas of composition CO+H₂. This syngas is too CO rich for utilisation in a FT reaction. However, if a third of the CO is converted to H₂ in a WGS-reactor, the composition is changed to CO+2 H₂, suitable for methanol synthesis.

In a preferred embodiment according to the invention, the syngas composition (a) used in step (i) is provided with or without using a shift reactor to produce hydrogen from CO and H₂O (CO+H₂O [symbol] CO₂+H₂). Using methanol as target intermediate (rather than sending the syngas directly to a FT-reactor), results in much lower syngas purification measures (lower capex and opex). This reaction can also be used in a PtL-pathway that uses a syngas produced by adding hydrogen to a stream of CO generated by decomposing (thermally or electrically) CO₂ to CO and oxygen as is the case in a CO₂/H₂O co-electrolysis pathway.

In a preferred embodiment according to the invention, the syngas composition (a) used in step (i) is obtained from a source selected from the group consisting of natural gas, coal, biomass, other hydrocarbon feedstocks, syngas obtained by reaction with steam (steam reforming), syngas obtained from carbon dioxide (dry reforming) or oxygen (partial oxidation or autothermal reforming) of carbon sources, syngas obtained from waste-to-energy gasification facilities, preferably from biomass sources.

According to this embodiment, it is preferred that the syngas composition (a) used in step (i) is supplemented by hydrogen.

The present invention can also be applied in steel manufacturing. The DRI (direct reduction of iron oxide) route uses a dry reforming of methane (CO₂ from the steel furnace flue gas and methane from natural gas) to produce syngas that is then used as reducing gas for iron synthesis in the shaft furnace:

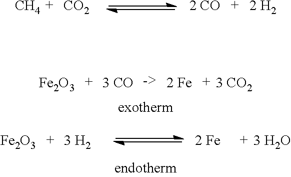

The state-of-the-art DRI process feeds the syngas at 950° C. into the shaft furnace containing the iron oxide. There, the hydrogen reacts faster, but at higher temperature (T>700° C.) than CO. The higher temperature requirement is caused by the enthalpy difference (616 kWh/t iron) and the fact that steam oxidises iron back to iron oxide. At T=700° C. iron formation is sufficiently favoured for the reaction to proceed.

Decomposition of dry and CO₂ free methanol according to the present invention generates a syngas with a H₂ content of 65% to 70%, preferably of 65.5% to 68% and more preferably of 66% to 67% and a CO content of 20% to 40%, preferably of 24% to 38%, more preferably of 28% to 35% and most preferably of 32% to 34% after recycling of the liquid phase (mainly methanol). The syngas stream can then be separated into a CO rich and a H₂ rich stream as described above. Although it is possible to use the H₂ rich stream for the DRI process of steel manufacturing according to the present efforts of the global steel industry to decarbonise steel manufacturing, it is actually energetically more efficient to use the CO rich stream instead for this purpose (if the methanol used qualifies for Net Zero carbon emissions).

If the CO rich stream is fed into the shaft furnace to react with the iron oxide at a temperature of 300° C. to 800° C., preferably at 400° C. to 700° C., more preferably at 450° C. to 600° C. and most preferably at 500° C. to 550° C., then the hydrogen of the H₂ rich stream can be used for other purposes, e.g. fed into a pipeline based public network or to operate a hydrogen power plant for the generation of electricity.

It is within the scope of the present invention to operate the DRI steel plant with a syngas of flexible CO and H₂ composition. The H₂ content then corresponds with the H₂ demand of the hydrogen power plant. At times of high electricity demand, more hydrogen is sent to the hydrogen power plant and less into the steel furnace. Correspondingly, more CO is needed for steel manufacturing and more methanol has to be decomposed. Also, the operating temperature of the shaft furnace has to be adjusted.

Alternatively, the CO rich stream can be operated as CO pure stream and the hydrogen used to convert the CO₂ flue gas from the DRI process for methanol synthesis in a DHC (direct hydrogenation of CO₂) process. The net methanol demand of the present invention decreases accordingly.

More alternatively, it is possible to operate the methanol decomposer with a sub-stoichiometric amount of steam. Then, the water reacts with methanol to form CO₂ and hydrogen

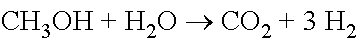

The excess methanol decomposes to CO and hydrogen

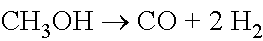

The total equation of methanol decomposition in this case becomes

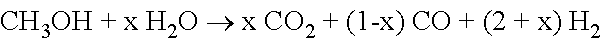

In this mode, additional hydrogen can be co-produced while providing CO to an industrial process (e.g. steel production).

Within this process, it is possible to substitute the missing hydrogen for the recycling of all the CO₂ back to methanol by electrolysis, enzymatic hydrogenation of CO₂, hydrogen from chemical sources, natural hydrogen, steam reforming of RNG or other sources of hydrogen. It is also possible to permanently store the excess CO₂ (CCS: carbon capture and storage) or to send the CO₂ back to the methanol production site (circular economy).

The catalysts used in the thermal-catalytical methanol decomposer are selected from compounds of the elements Rh, Pd, Pt, Ir, Ru, Fe, Zn, Co, Ni, Cu and Mn, preferably from the elements Zn, Cu, Fe, Pt, Pd and Rh, more preferably from the elements Zn, Cu and Fe and most preferably catalysts containing either ZnO, a combination of ZnO and Cu or iron oxides. The methanol decomposer can be a shaft furnace. The methanol is fed into the shaft furnace as a hot methanol vapour instead of the hot syngas used in the state-of-the-art DRI process.

The hot methanol vapour is thermal-catalytically decomposed by the iron oxide (Fe₂O₃, Fe₃O₄ or other iron oxide containing ores) to a syngas which then in turn reduces the iron oxide to iron (HBI; hot briquetted iron) according to the state-of-the-art DRI process. The iron oxide causes the dehydrogenation of methanol to formaldehyde, which then thermally decomposes to CO and hydrogen

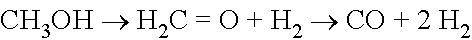

The process is operated at temperatures between 500° C. and 950° C., preferably between 550° C. and 850° C., more preferably between 550° C. and 700° C. and most preferably between 550° C. and 650° C.

In a specific aspect, the invention is directed at a process for the thermal catalytic decomposition of methanol into carbon monoxide and hydrogen, said process comprising the step of reacting methanol in a methanol decomposition reactor, optionally under one or more of the following conditions:
(a) a temperature in the range of 100° C. to 400° C., preferably of 200° C. to 350° C., more preferably of 220° C. to 300° C. and most preferably of 240° C. to 270° C.
(b) a pressure in the range 10 bar to 70 bar, preferably 20 bar to 50 bar, more preferably 35 bar to 45 bar,
(c) the use of dried methanol comprising less than 10 wt.-% water, preferably less than 1 wt.-% water, more preferably less than 0.5 wt.-% water, most preferably less than 0.2 wt.-% water,
(d) a catalyst selected from the group consisting of compounds of the elements Rh, Pd, Pt, Ir, Ru, Fe, Zn, Co, Ni, Cu and Mn, preferably from the elements Zn, Cu, Fe, Pt, Pd and Rh, more preferably from the elements Zn, Cu and Fe and most preferably catalysts containing either ZnO, a combination of ZnO and Cu or iron oxides, Cu/ZnO, Cu/ZnO/Al₂O₃, doped Cu/ZnO, doped Cu/Zn/Al₂O₃, Fe₂O₃ or Fe₃O₄,
optionally separating at least part of the resulting mixture into CO enriched and H₂ enriched gas streams, e.g. by means of a membrane separation unit,
optionally recombining the CO enriched and H₂ enriched gas streams with each other and/or the main stream resulting from the decomposition unit.

This means the invention is also directed to a process comprising a decomposition step of methanol as described as step (vii) of the process for the production of syngas mandatorily comprising step (i) and step (vii) above and below, with the difference that the methanol can be from any source and is not restricted to the production from a syngas composition.

The process for the thermal catalytic decomposition of methanol into carbon monoxide and hydrogen comprising the step of reacting methanol in a methanol decomposition reactor comprises the steps of
(vii) Decomposing methanol into carbon monoxide and hydrogen by thermal catalytic decomposition in a step of reacting methanol in a methanol decomposition reactor,
(viii) Optionally subjecting the syngas (b) to a separation step in a separation unit, wherein CO enriched and H₂ enriched streams are obtained,
(ix) Optionally subjecting the syngas (b) or any CO enriched and/or H₂ enriched streams obtained therefrom to a Fischer Tropsch (FT) synthesis step,
(x) Optionally using the syngas (b) or any CO enriched and/or H₂ enriched streams obtained therefrom for the reduction of any iron oxide to iron, wherein optionally the process is performed by two or more individual parties.

Accordingly, all features and process characteristics as described for step (vii) and the optional steps (viii) to (x) up to here and below also apply to the process for the thermal catalytic decomposition of methanol of this aspect, except for the methanol used in step (vii) is not necessarily produced from a syngas composition.

### DESCRIPTION OF THE FIGURES

FIG. 1: Design of a PtL-plant according to the present invention. The methanol synthesis-decomposition unit is displayed inside the black-circled box.

FIG. 2: Design of a PtL-plant according to the present invention. The methanol synthesis is performed by DHC (direct hydrogenation of CO₂).

FIG. 3: Design of a PtL-plant according to the present invention. The syngas composition is regulated by a syngas separation unit.

FIG. 4: Design of a PtL-plant according to the present invention. The methanol is synthesised from CO₂ and hydrogen (DHC). The syngas composition is regulated by a syngas separation unit.

FIG. 5: Schematic view of a membrane filtering unit used in the invention.

FIG. 6: Possible Mechanism of the formation of methanol from CO₂.

FIG. 7: Possible Mechanism of the formation of syngas from methanol.

FIG. 8: Design of a prior art syngas-to-methanol reactor and a methanol-to-syngas reactor according to the present invention.

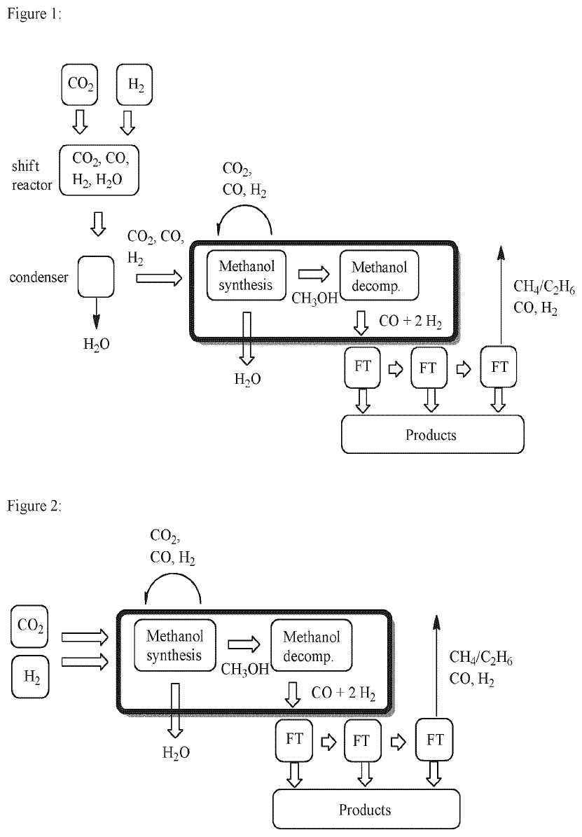

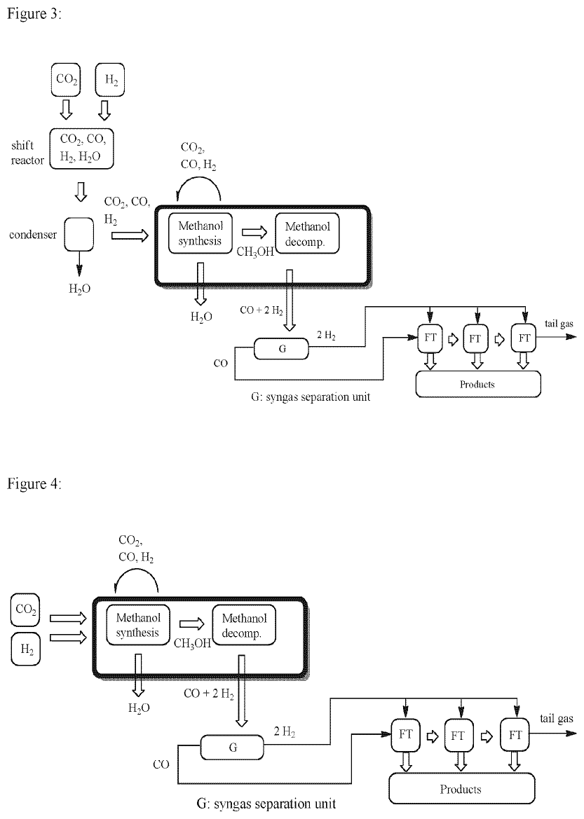

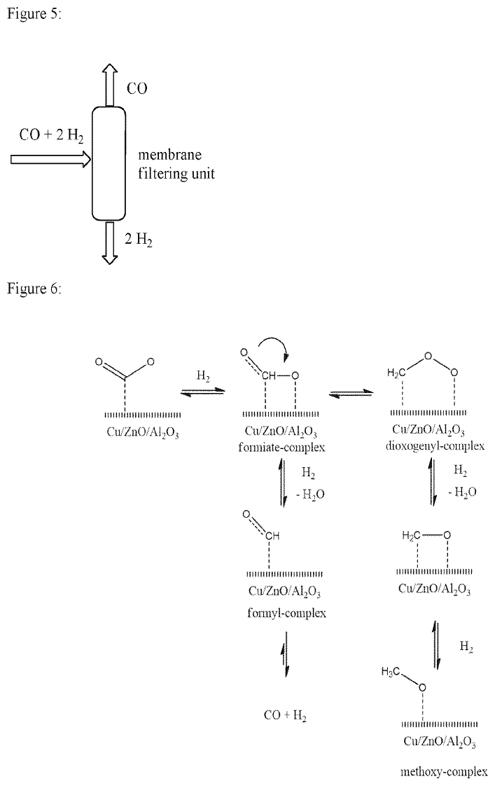

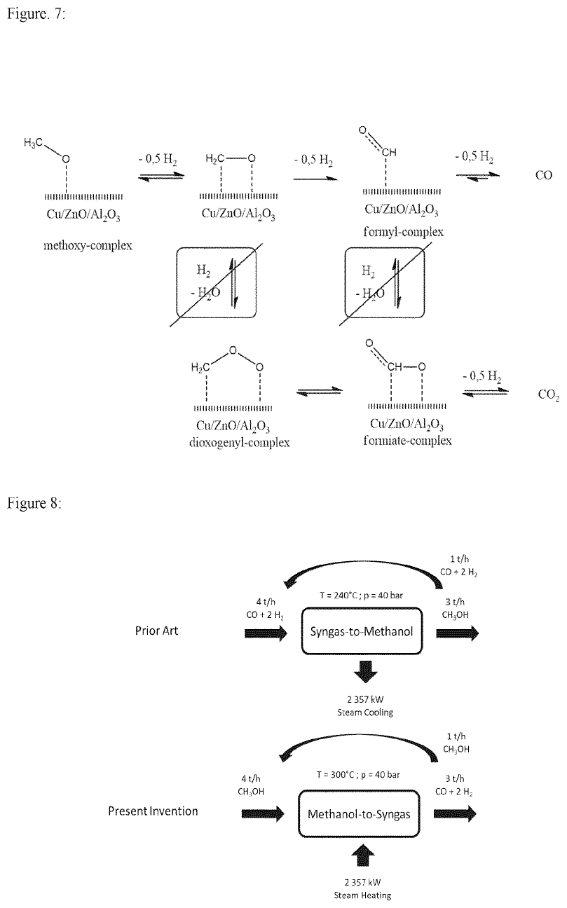

### FURTHER PREFERRED EMBODIMENTS

### Embodiment 1

In a preferred first embodiment of the invention a gas mixture containing carbon dioxide and at least the same molar amount of hydrogen is fed into a RWGS (reverse water gas shift) reactor and shifted to a syngas containing at least carbon monoxide, hydrogen and water.

The water-gas shift reaction (WGSR) describes the reaction of carbon monoxide and water vapor to form carbon dioxide and hydrogen:

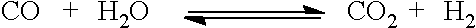

or as RWGS (reverse water gas shift reaction) the other way around:

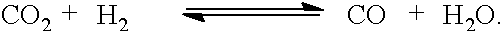

Using an excess of hydrogen in such reverse water gas shift reaction will lead to a syngas composition comprising CO and H₂.

As the RWGS reaction is an equilibrium reaction, not all of the CO₂ reacts and the product syngas will contain some CO₂. The resulting syngas is dried by condensing the water out and then fed into a methanol synthesis reactor, where the syngas is converted to methanol e.g. according to the following stoichiometry, which reflects the maximum CO₂ content for methanol synthesis using modern Cu/ZnO/Al₂O₃ catalysts:

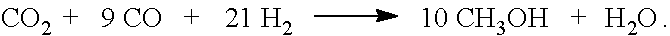

The methanol is separated, dried and ultimately fed again into a methanol decomposition reactor used in step (vii).

In the methanol decomposition reactor, the methanol is thermally decomposed (with the use of catalysts) to form syngas, in particular, of the composition CO+2 H₂, which process is described in more detail below.

The syngas stream (b) from the methanol decomposition step (vii) optionally can be fed into a separation unit. The separation unit for example divides the syngas stream into a CO rich stream and a hydrogen rich stream. One way to achieve this is to utilise a membrane filtering unit that with the aid of a pressure drop across a membrane, lets the fast-moving gas (here hydrogen) pass through the membrane while the slow moving gas (here CO) does not pass through the membrane and leaves the unit through another exit. After the separation unit, the two gas streams can be re-mixed in a way so that a CO rich syngas stream of preferred composition (e.g. of a molar ratio CO/H₂ of about 1:1 to 1:4, preferably 1:1.5 to 1:3, more preferably 1:1.6 to 1:2, most preferably of about 1:1.7 to 1:1.8) results.

The syngas of a preferred composition is then preferably fed into a string of one or more, preferably two or more, more preferably 2-3 FT-reactors. The flue gas from the first FT-reactor is very CO enriched and is mixed with enough of the hydrogen rich gas stream from the separation unit to reach again a preferred composition (e.g. of a molar ratio CO/H₂ of about 1:1 to 1:4, preferably 1:1.5 to 1:3, more preferably 1:1.6 to 1:2, most preferably of about 1:1.7 to 1:1.8) before it enters the second FT-reactor. The procedure is repeated for the third and any subsequent FT-reactor, if required.

### Embodiment 2

The RWGS reactor in embodiment 1 is replaced by a DHC-reactor (DHC: direct hydrogenation of CO₂). A mixture of 25% (v/v) CO₂ and 75% (v/v) H₂ is fed into the reactor. The products methanol and water are condensed out and are fed into a distillation apparatus and the methanol is distilled from the water. The unreacted feedstock gases (CO₂ and H₂) are topped up and recycled back into the DHC-reactor.

### Embodiment 3

The RWGS-reactor in embodiment 1 is replaced by a biomass gasifier. The product gas mixture from a biomass gasifier containing at least carbon monoxide, an insufficient amount of hydrogen and typically some CO₂ is fed-together with additional hydrogen (to convert all CO and usually contained CO₂ into methanol)-into a methanol synthesis reactor according to step (i). In the methanol synthesis reactor, the syngas is converted to methanol, for example according to the following equation, which reflects the maximum CO₂ content for methanol synthesis using modern Cu/ZnO/Al₂O₃ catalysts:

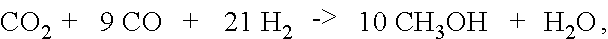

The methanol is separated, dried and ultimately (optionally after further purification, storage or transport to another place) fed into a methanol decomposition reactor used in step (vii). In the methanol decomposition reactor, the methanol is thermally decomposed (with the use of catalysts) to form syngas, in particular, of the composition CO+2 H₂. The syngas stream can then be fed into a separation unit as described before. The separation unit divides the syngas stream into a CO rich stream and a hydrogen stream. One way to achieve this is to utilise a membrane filtering unit that with the aid of a pressure drop across a membrane, lets the fast moving gas (here hydrogen) pass through the membrane while the slow moving gas (here CO) does not pass through the membrane and leaves the unit through another exit. After the separation unit, the two gas streams can be re-mixed in a way so that a CO poor syngas stream of preferred composition (e.g. of a molar ratio CO/H₂ of about 1:1 to 1:4, preferably 1:1.5 to 1:3, more preferably 1:1.6 to 1:2, most preferably of about 1:1.7 to 1:1.8) results, as described before.

The syngas of a preferred composition is then preferably fed into a string of one or more, preferably two or more, more preferably 2-3 FT-reactors. The flue gas from the first FT-reactor is very CO enriched and is mixed with enough of the hydrogen rich gas stream from the separation unit to reach again a preferred composition (e.g. of a molar ratio CO/H₂ of about 1:1 to 1:4, preferably 1:1.5 to 1:3, more preferably 1:1.6 to 1:2, most preferably of about 1:1.7 to 1:1.8) before it enters the second FT-reactor. The procedure is repeated for the third and any subsequent FT-reactor, if required as described before.

### Embodiment 4

Dry and CO₂ free methanol is fed into a methanol decomposer according to the present invention. From the stream of syngas, a stream of hydrogen is separated and sent downstream for further use. The remaining CO containing stream is fed into a shaft furnace, where it reduces iron oxides to iron (HBI; hot briquetted iron).

The CO containing stream is a syngas of composition CO+ (2-x) H₂, where x is the amount of hydrogen removed from the original amount of hydrogen. The amount x is variable and can be adjusted according to the changing hydrogen needs of external consumers. Thus, embodiment 4 serves as a flexible hydrogen source and has the same effect as a hydrogen storage device with the advantage that it does not rely on extensive hydrogen transport and storage infrastructure.

### Embodiment 5

The facilities of embodiment 4 with the alterations that the catalyst is a ZnO containing catalyst and an amount of water, preferably 1 wt.-% to 90 wt.-% water based on the amount by weight of methanol, more preferably 10 wt.-% to 85 wt.-%, even more preferably 20 wt.-% to 70 wt.-%, most preferably 20 wt.-% to 50 wt.-% water is added to the methanol. The water will decompose the methanol to CO₂ and hydrogen with the effect that more methanol is decomposed to produce the same amount of CO and at the same time, an increased mount of hydrogen can be distributed to external consumers.

The hydrogen distribution capacity has increased compared to embodiment 4.

The CO₂ that is co-produced with the CO will pass through the shaft furnace without interfering with the steel production process.

### Embodiment 6

The facilities from embodiment 4, but without the methanol decomposer. The methanol is vapourised and fed hot into the shaft furnace. There, it is decomposed to CO and hydrogen by the iron oxides acting as catalysts. The reducing gases CO and hydrogen then proceed to reduce the iron oxides to HBI.

The process is operated at temperatures between 500° C. and 950° C., preferably between 550° C. and 850° C., more preferably between 550° C. and 700° C. and most preferably between 550° C. and 650° C.

At high temperatures of the iron oxide bed, both CO and hydrogen react. At high temperatures of the iron oxide bed, only CO reacts and most of the hydrogen passes through unchanged. The hydrogen can be separated from the flue gas and sent downstream for further use. In embodiment 6, the shaft furnace itself acts as methanol decomposer according to the present invention.

### Mechanism of Methanol Synthesis

Regarding the direct hydrogenation of CO₂ to methanol, it follows the equation:

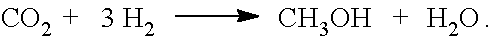

In accordance with the process of the invention the methanol can be decomposed again to syngas according to equation:

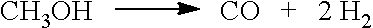

with the equilibrium constant depending on temperature and pressure.

It is well known to the expert in the field that methanol can be steam reformed to obtain hydrogen. However, the presence of water in this reaction oxidises the methanol to CO₂ (CH₃OH+H₂O→CO₂+3 H₂). For this reason, the present invention proposes the use of dry methanol, which has a higher yield, lower energy consumption and no CO₂ formation. Methanol formation is generally favoured at high pressure and low temperature (irrespective whether CO or CO₂ is the starting material) and occurs in a temperature window between T=100° C. and T=400° C. At the high end of this temperature window, the catalyst can be permanently damaged by sintering.

The corresponding methanol synthesis from syngas (hydrogenation of CO)

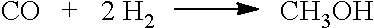

is greatly aided by addition of CO₂ and proceeds very reluctantly in the absence of CO₂. For this reason, a small amount of CO₂ is preferably added or contained in the syngas composition (a) to start the reaction to form methanol. In the process, some H₂O is formed, which may temporarily poison the catalyst. At higher water concentrations (depending on the exact catalyst composition, but with a current maximum concentration of about 10%), the reaction is terminated. Termination occurs because the methanol forming reaction CO₂+3 H₂→CH₃OH+H₂O is in equilibrium with the methanol decomposition reaction CH₃OH+H₂O→CO₂+3 H₂.

The generally agreed mechanism is depicted in FIG. 6.

The key step is the formation of the deoxogenyl-complex by a synchronized C—O bond breakage, formyl rotation and O—O bond formation sequence. If the delicate last step, O—O bond formation, fails to eventuate, a formyl-complex is formed that is unstable and decomposes to CO+H₂.

Although the agreed mechanism would suggest the reversibility of this step in the presence of water, it is not discussed in the literature. This is maybe not surprising realizing that normally dry syngas is utilized for methanol synthesis. Dry syngas fails to react to methanol, unless CO₂ is added.

It is not the CO₂ that enables methanol synthesis, but the water that is formed from it, when the CO₂ is hydrogenated.

Likewise, when dry and CO₂ free methanol is thermally decomposed to syngas (CH₃OH→CO+2 H₂) as part of the present invention, the syngas cannot react back to methanol. There is no water present that would facilitate formation of the deoxogenyl-complex from the H₂C—O entity or the formiate complex from the formyl-complex by addition of water as part of the sequence of the reaction mechanism (FIG. 7). The formyl-complex, once formed, can only decompose back to CO and hydrogen. Water acts as a co-catalyst in the synthesis of methanol and its absence converts the apparent equilibrium reaction into a quasi-irreversible reaction that can only decompose methanol into syngas, but no longer converts syngas into methanol. It should be mentioned that the presence of water and/or CO₂ does not prevent the decomposition of methanol to syngas, but it allows the re-formation of methanol during methanol decomposition. This lowers the single pass rate (SPR) and thus the syngas yield. As a consequence, during the process of the current invention, much more syngas is formed at lower temperature and/or higher pressure than calculated by conventional computer simulations, if dry and CO₂ free methanol is used.

In a preferred embodiment of the invention the methanol decomposed in step (vii) is preferably dried and CO₂ free methanol, preferably comprising less than 10 wt.-% water, preferably less than 1 wt.-% water, more preferably less than 0.5 wt.-% water, most preferably less than 0.2 wt.-% water. The specifications of dry and CO₂ free methanol are given by standard methanol data sheets such as they are provided by the Methanol Institute (Methanol-Technical-Data-Sheet.pdf). Specifically, for the properties of dry and CO₂ free methanol reference is made to the “IMPCA Methanol Reference Specifications” Version 9, dated 10 Jun. 2021 and the methods referred to in there. Dry and CO₂ free methanol can be produced by distillation and verified by standard spectroscopic means and density measurements (Lange's Handbook of Chemistry, 10th ed. and CRC Handbook of Chemistry and Physics 44th ed.). The catalyst is a commercial Cu/ZnO/Al₂O₃ catalyst.

Optionally, the methanol decomposer is used to produce hydrogen in addition to the amount of CO required. In this case, 1 wt.-% to 90 wt.-% water based on the amount by weight of methanol, preferably 10 wt.-% to 85 wt.-%, more preferably 20 wt.-% to 70 wt.-%, most preferably 20 wt.-% to 50 wt.-% water in addition to methanol is used.

Although the above reaction mechanism of the formation of methanol from CO₂ is generally known and discussed in academic circles (Chemical Society Reviews, 2020, 1385), its consequences are generally unknown. The academic literature has identified the formation of the dioxogenyl-complex from the formiate-complex as the rate determining step and even sees the failure to form the dioxogenyl-complex as the main reason for the formation of the formyl-complex (and subsequently CO in a RWGS analogous reaction), but fails to realize that it is the absence of water—and not the absence of CO₂—that prevents the direct hydrogenation of CO (syngas-to-methanol) reaction in conventional methanol synthesis from syngas.

In accordance with the present invention, the methanol decomposition in step (vii) is preferably carried out with dried and CO₂ free methanol. It comprises for example less than 10 wt.-% water, preferably less than 1 wt.-% water, more preferably less than 0.5 wt.-% water, most preferably less than 0.2 wt.-% water.

The process of the methanol decomposition to form the syngas (b) of the invention thus allows, in particular:
The efficient decomposition of methanol to syngas, and
The generation of syngas at low reaction temperatures.

Since the decomposition of dry and CO₂ free methanol to syngas according to the present invention is not an equilibrium reaction, as described in the literature, but an irreversible process, the decomposition proceeds uni-directional without reformation of methanol. It is therefore possible to decompose dry and CO₂ free methanol at low temperatures (T<350° C., preferably <250° C. and more preferably at T<200° C.) in a reactor with a longer catalyst bed or with a lower feedstock velocity than the respective syngas-to-methanol reactor for methanol synthesis. In that way, catalyst degradation by sintering (at T>300° C.) and problems with heat transfer (the kinetics of heat transfer to the catalyst bed and/or the methanol feedstock) can be avoided.

Unreacted methanol can be condensed, separated from the syngas and sent back into the reactor.

### Thermal-Catalytical Decomposition of Methanol

The formation of methanol and its thermal-catalytical decomposition to syngas again in accordance with the process of the invention is described in the following in further detail.

### Synthesis of Methanol

Methanol is produced preferably either from syngas (StM: syngas-to-methanol) or from CO₂ (DHC direct hydrogenation of CO₂). Both technologies preferably use a pipe-bundle reactor with the same or similar catalyst (Cu/ZnO/Al₂O₃) filling and the same or similar reaction conditions (pressure of 10 bar to 70 bar, preferably 30 bar to 50 bar and more preferably 40 bar; temperature of 100° C. to 400° C., preferably of 200° C. to 350° C., more preferably of 220° C. to 300° C. and most preferably of 240° C. to 270° C.) and intensive cooling to remove the process heat.

A key step in the DHC route is the recycling of the unreacted feedstock gases with a SPR (single pass rate) of about 10% to 15%, depending on the actual Cu/ZnO/Al₂O₃ catalyst used. The products methanol and water are removed by condensation (cooling of the product gas) and the residual gas (CO₂ and H₂) topped up prior to refeeding it into the synthesis reactor. The cooling cycle can be connected to a heat exchanger that operates as a secondary cooling cycle.

The DHC-process can be reversed and is then known as the MSR (methanol-steam-reforming) process. Theoretically, there are two possible mechanisms for the MSR-process

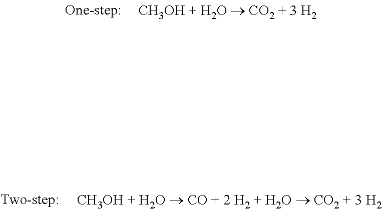

Despite extensive experimental research, no traces of CO as the intermediate of the two-step mechanism has ever been observed (Liu et al). It is therefore generally recognised that the MSR-process proceeds according to the one-step mechanism.

The WGS (water gas shift) reaction is also performed with the MSR-reactor, but from CO rather than methanol as starting material

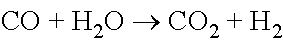

It was the WGS-reaction that has given rise to the assumption that the MSR-reaction might proceed by decomposition followed by WGS of the CO to CO₂. This assumption was disproven. However, the present invention shows that despite the teachings of the state-of-the-art, dry methanol (methanol in the absence of water) can be decomposed to CO+2 H₂. What is more, as water is consumed in the MSR-reaction, dry decomposition of methanol (to CO+2 H₂) occurs after all the water is consumed, when wet methanol is used.

### Prior Art Reactor

It is known that the synthesis of methanol is a temperature dependent equilibrium reaction with decomposition dominating at higher temperatures (see FIGS. 6 and 7). The two standard prior art reactors (StM and DHC) differ in their SPR (StM: 65-85%, DHC: 10-15%), their process heat (StM: ΔH=−90.8 KJ/mol; DHC: ΔH=−49.6 KJ/mol), the capacity of their cooling system and their recyclation system. The difference in the cooling system reflects the higher process heat liberated by the StM-reactor and the difference in the recycling system reflects the much larger amount of unreacted feedgas recycled by the DHC reactor.

For the purpose of the methanol decomposer (methanol-to-syngas MtS reactor) of the present invention, a hybrid system that uses components of both methanol synthesis reactors is utilised and additional modifications introduced.

### Methanol Decomposer of the Present Invention

The standard commercial StM-reactor is fed with gaseous methanol. The pipe-bundle catalyst bed is filled with the same commercial Cu/ZnO/Al₂O₃ catalyst as the original StM-reactor. The temperature of the pipe-bundle catalyst bed is regulated with the same steam cycle, but at a higher temperature.

The methanol decomposition reaction: CH₃OH→CO+2 H₂ is endotherm and requires heating, whereas the methanol synthesis reaction: CO+2 H₂→CH₃OH is exotherm and requires the same amount of cooling. For this reason, the StM-reactor is chosen as the base and the steam cycle of the present invention is equipped with a heat exchanger and a standard steam heating system outside the reactor. The expert in the art also knows of ways to incorporate the heating system inside the reactor and an interior heating system is within the scope of the invention.

The decomposed methanol is a syngas of the composition CO+2 H₂ (defined by the chemical equation) and can be removed with the same recycling loop known from methanol synthesis by DHC (90% recycling of CO₂+3 H₂). Except this time, the gas phase is the product and the liquid phase (CH₃OH) is recycled back into the reactor. For this reason, the liquid methanol is brought into the gas phase and recycled back into the MtS-reactor; the syngas is sent downstream.

It is possible to use a standard MSR-reactor and operate it with dry and CO₂ free methanol. It then serves as a methanol decomposer according to the present invention, after due adjustment of the heating system.

### Reactor Modifications

The customary pipe-bundle reactor of both the DHC and the StM pathways has the pipe-bundle submerged in the cooling medium inside the pressure container of the reactor.

The cooling medium itself is either steam, thermal oil or liquid salt.

For safety reasons, the pressure limit of the cooling medium should be above the default pressure of the pipe-bundle and thus above the methanol synthesis operating pressure, which is usually between 10 bar and 70 bar, preferably between 20 bar and 60 bar and more preferably between 35 bar and 50 bar.

Switching the reactor from synthesis mode at a temperature of 200° C. to 400° C., preferably of 220° C. to 350° C., more preferably of 230° C. to 300° C. and most preferably of 240° C. to 270° C. to decomposition mode at a temperature range of 100° C. to 400° C., preferably of 200° C. to 350° C., more preferably of 220° C. to 300° C. and most preferably of 240° C. to 270° C. has consequences for the thermal fluid. In the case of steam, overheating the saturated steam from 250° C. to 350° C. increases the pressure to near the safety limit of 40 bar to 50 bar of the outer pressure wall. In the case of thermal oil and liquid salt, the material may not be thermally stable at 350° C. In both cases, the fluid can be replaced by a thermal oil or liquid salt of higher thermal stability. In most cases, retrofitting of a methanol synthesis reactor to decomposition mode will be possible with no or only minor modifications to the reactor itself.

### Embodiment 7

A commercially available pipe-bundle StM-reactor with a capacity of 4 t/h syngas (CO+2 H₂ with a CO₂ content of 3% v/v) and a SPR of 75% produces 3 t/h methanol at a reaction temperature of 240° C. and a pressure of 40 bar using a commercial Cu/Zn/Al₂O₃ catalyst. The process heat of 2,357 kWh is taken out of the reactor by a steam cooling cycle. The gaseous products consisting of 3 t/h methanol and 1 t/h syngas (CO+2 H₂ containing water and CO₂) are sent to a recyclization unit that cools the product gas until the water and the methanol condenses. The condensed liquid phase is removed from the recyclisation unit and sent to a distillation apparatus. The gaseous phase (CO, H₂, CO₂) is recycled, topped up to 4 t/h and fed back into the StM-reactor.

For the process of the present invention, the StM-reactor is retrofitted to serve as a MtS-reactor for the decomposition of methanol to syngas. A methanol storage tank is used to dispense 4 t/h dry and CO₂ free methanol to an evaporation, compression and heating unit to provide gaseous dry and CO₂ free methanol at the reaction pressure of 10 bar to 70 bar, preferably 20 bar to 50 bar, more preferably 35 bar to 45 bar and the reaction temperature of 100° C. to 400° C., preferably of 200° C. to 350° C., more preferably of 220° C. to 300° C. and most preferably of 240° C. to 270° C. The dry and CO₂ free methanol vapour is sent through the MtS-reactor where it is decomposed to 3 t/h syngas (CO+2 H₂) and a SPR of 75%. The necessary process heat is provided by a steam heating device that heats the steam in the steam cycle of the MtS-reactor at a point outside the reactor to the reaction temperature. For this purpose, the steam cooling cycle of the original StM-reactor is retrofitted with a heating device outside the reactor. During the decomposition of the methanol and while the steam flows through the reactor, the methanol decomposition reaction draws heat from the steam and causes the steam to cool down. Subsequently, the heating device heats the steam back up to its default value.

The MtS-reactor is retrofitted with a new recycling unit that is very similar to the original one in as much as it condenses the methanol (CH₃OH) and separates the liquid methanol from the gaseous syngas (CO+2 H₂). The methanol is recycled back to the methanol evaporation, compression and heating unit, where it is topped up to 4 t/h. The difference in the recycling unit is the capacity for processing the liquid phase (drops from 75% or 3 t/h to 25% or 1 t/h) whereas the gaseous phase increases accordingly (from 25% or 1 t/h to 75% or 3 t/h). The other difference is that now the liquid phase is recycled and the gaseous phase sent downstream. The retrofitted recycling unit resembles in its relative capacities that of an equivalent DHC-reactor for methanol synthesis.

1. A process for the production of syngas by thermal catalytic decomposition of methanol produced from a mixture comprising at least a carbon oxide (CO and/or CO₂) and hydrogen.

2. A process for the production of syngas according to claim 1, comprising the steps of:
(i) Producing methanol from a syngas composition (a),
(ii) Optional separation of the methanol,
(iii) Optional drying of the methanol,
(iv) Optional purification of the methanol,
(v) Optional storage of the methanol,
(vi) Optional transport of the methanol,
(vii) Producing syngas (b) from methanol obtained in step (i) by thermal catalytic decomposition,
(viii) Optionally subjecting the syngas (b) to a separation step in a separation unit, wherein at least CO enriched and H₂ enriched streams are obtained,
(ix) Optionally subjecting the syngas (b) or any CO enriched and/or H₂ enriched streams obtained therefrom to a Fischer Tropsch (FT) synthesis step.
(x) Optionally using the syngas (b) or any CO enriched and/or H₂ enriched streams obtained therefrom for the reduction of any iron oxide to iron,
wherein optionally the process is performed by two or more individual parties.

3. A process according to claim 2, wherein the syngas composition (a) used in step (i) comprises a carbon oxide, selected from CO and CO₂, and hydrogen.

4. A process according to any of the previous claims 2 and 3, wherein the syngas composition (a) used in step (i) has a molar ratio of carbon oxide to hydrogen in the range of 1:4 to 1:1 preferably 1:3 to 1:2.

5. A process according to any of the previous claims 2 to 4, wherein the carbon oxide in the syngas composition (a) used in step (i) comprises carbon dioxide (CO₂).

6. A process according to any of the previous claims 2 to 5, wherein the syngas composition (a) used in step (i) is obtained from subjecting carbon dioxide and hydrogen to a reverse water gas shift reaction (CO₂+H₂ [symbol] CO+H₂O).

7. A process according to any of the previous claims 2 to 6, wherein the syngas (b) obtained in step (vii) comprises carbon monoxide and hydrogen, in a molar ratio of more than about 1:1 to about 1:3, preferably of about 1:1 to about 1:2.

8. A process according to any of the previous claims 2 to 7, wherein the syngas (b) obtained in step (vii) is subjected to a separation step in a separation unit, wherein CO enriched or H₂ enriched streams are obtained, which optionally can be used directly in subsequent processes or after recombination with the main stream from the methanol decomposition unit used in step (vii), in particular, to adjust a certain CO/H₂ molar ratio.

9. A process according to any of the previous claims 2 to 8, wherein the syngas (b) obtained in step (vii) is subjected to a separation step in a separation unit, wherein a stream of CO is obtained that is used to polymerise CO to a polyketone.

10. A process according to any of the previous claims 2 to 9, wherein the syngas (b) obtained in step (vii) is used to reduce an iron oxide to iron.

11. A process according to any of the previous claims 2 to 10, wherein the syngas (b) obtained in step (vii) is subjected to a separation step in a separation unit, wherein CO enriched or H₂ enriched streams are obtained, whereby either the one or the other is used for the reduction of iron oxide to iron, and wherein optionally
(a)—the H₂ content of the CO rich stream used for iron oxide reduction is modified according to an external hydrogen demand, and/or
(b)—the H₂ rich stream is used to export hydrogen into a grid and/or a storage tank, and/or
(c)—the H₂ rich stream is used for electricity generation, and/or
(d)—the H₂ rich stream is used to convert part of the CO₂ from iron oxide reduction to produce methanol.

12. A process according to any of the previous claims 2 to 11, wherein the CO enriched or H₂ enriched streams obtained are combined with a stream selected from:
the syngas stream resulting from the methanol decomposing unit used in step (vii) to provide CO or H₂ enriched streams of a certain molar ratio, and
any stream leaving a FT reactor in the FT synthesis step (ix), to provide a combined CO/H₂ stream preferably having a molar ratio CO/H₂ of about 1:1 to 1:4, preferably 1:1.5 to 1:3, more preferably 1:1.6 to 1:2, most preferably of about 1:1.7 to 1:1.8.

13. A process according to any of the previous claims 2 to 12, wherein the syngas (b) obtained in step (vii) is transferred into a FT synthesis unit, wherein the syngas (b) is subjected to a FT synthesis step (ix), providing higher molecular products having two or more, preferably three or more carbon atoms selected from the group consisting of alkanes, alkenes or alcohols.

14. A process according to any of the previous claims 2 to 13, wherein the syngas (b) obtained in step (vii) is transferred to a FT synthesis unit consisting of one or more, preferably 2 or more, more preferably 2 to 3 FT reactors.

15. A process according to any of the previous claims 2 to 14, wherein the syngas (b) obtained in step (vii) is transferred to a FT synthesis unit, consisting of one or more, preferably two or more FT reactors and wherein a CO enriched stream leaving a FT reactor is recombined with a H₂ enriched stream preferably resulting from the separation step (viii), so as to preferably provide a stream having a molar ratio CO/H₂ of about 1:1 to 1:4, preferably 1:1.5 to 1:3, more preferably 1:1.6 to 1:2, most preferably of about 1:1.7 to 1:1.8.

16. A process according to any of the previous claims 2 to 15, wherein at least part of the process heat of the methanol producing step (i) and/or of the FT synthesis step (ix) are transferred to the methanol decomposition step (vii).

17. A process according to any of the previous claims 2 to 16, wherein the syngas composition (a) used in step (i) is obtained from a source selected from the group consisting of natural gas, coal, biomass, other hydrocarbon feedstocks, syngas obtained by reaction with steam (steam reforming), syngas obtained from carbon dioxide (dry reforming) or oxygen (partial oxidation or autothermal reforming) of carbon sources, syngas obtained from waste-to-energy gasification facilities, preferably from biomass sources.

18. A process according to any of the previous claims 2 to 17, wherein the syngas composition (a) used in step (i) is supplemented by hydrogen.

19. A process according to any of the previous claims 2 to 18, wherein the syngas composition (a) used in step (i) is provided with or without using a shift reactor to produce hydrogen from CO and H₂O (CO+H₂O [symbol] CO₂+H₂).

20. A process according to any of the previous claims 2 to 19, wherein step (ix) is carried out and wherein the tail gas of the FT-reactor(s) is combusted to provide heat for the methanol decomposition step (vii), and optionally the CO₂ from the combustion gas is recycled into the methanol synthesis step (i), wherein the tail gas preferably comprises CO₂, H₂ and CO.

21. A process according to any of the previous claims 2 to 20, wherein step (ix) is carried out and wherein the tail gas of the FT-reactor(s) is recycled back into step (i), wherein the tail gas preferably comprises CO₂, H₂ and CO.

22. A process according to any of the previous claims 2 to 21, wherein the syngas (b) obtained in step (vii) is used for the production of hydrogen, ammonia, synthetic hydrocarbons for use as a fuel or lubricant, in particular via the Fischer-Tropsch process.

23. A process according to any of the previous claims 2 to 22, wherein step (vii) is carried out with one or more of the following conditions:
(a) a temperature in the range of 100° C. to 400° C., preferably of 200° C. to 350° C., more preferably of 220° C. to 300° C. and most preferably of 240° C. to 270° C.,
(b) a pressure in the range of 1 bar to 70 bar, preferably 20 bar to 50 bar, more preferably 35 bar to 45 bar,
(c) the use of dried methanol comprising less than 10 wt.-% water, preferably less than 1 wt.-% water, more preferably less than 0.5 wt.-% water, most preferably less than 0.2 wt.-% water,
(d) a catalyst selected from the group consisting of compounds of the elements Rh, Pd, Pt, Ir, Ru, Fe, Zn, Co, Ni, Cu and Mn, preferably from the elements Zn, Cu, Fe, Pt, Pd and Rh, more preferably from the elements Zn, Cu and Fe and most preferably catalysts containing either ZnO, a combination of ZnO and Cu or iron oxides, in particular Cu/ZnO, Cu/ZnO/Al₂O₃, doped Cu/ZnO, doped Cu/Zn/Al₂O₃, Fe₂O₃ or Fe₃O₄.

24. A process for the thermal catalytic decomposition of methanol into carbon monoxide and hydrogen, said process comprises the step of reacting methanol in a methanol decomposition reactor, optionally under one or more of the following conditions:
(a) a temperature in the range of 100° C. to 400° C., preferably of 200° C. to 350° C., more preferably of 220° C. to 300° C. and most preferably of 240° C. to 270° C.,
(b) a pressure in the range 10 bar to 70 bar, preferably 20 bar to 50 bar, more preferably 35 bar to 45 bar,
(c) the use of dried methanol comprising less than 10 wt.-% water, preferably less than 1 wt.-% water, more preferably less than 0.5 wt.-% water, most preferably less than 0.2 wt.-% water,
(d) a catalyst selected from the group consisting of compounds of the elements Rh, Pd, Pt, Ir, Ru, Fe, Zn, Co, Ni, Cu and Mn, preferably from the elements Zn, Cu, Fe, Pt, Pd and Rh, more preferably from the elements Zn, Cu and Fe and most preferably catalysts containing either ZnO, a combination of ZnO and Cu or iron oxides, Cu/ZnO, Cu/ZnO/Al₂O₃, doped Cu/ZnO, doped Cu/Zn/Al₂O₃, Fe₂O₃ or Fe₃O₄, optionally separating at least part of the resulting mixture into CO enriched and H₂ enriched gas streams, e.g. by means of a membrane separation unit,
optionally recombining the CO enriched and H₂ enriched gas streams with each other and/or the main stream resulting from the decomposition unit.

25. A process according to any of the previous claims 2 to 24, wherein dried methanol, preferably methanol comprising less than 10 wt.-% water, more preferably less than 1 wt.-% water, even more preferably less than 0.5 wt.-% water, most preferably less than 0.2 wt.-% water, is used in the methanol decomposition step (vii) and fed into the methanol decomposition unit, and wherein the reaction is carried out at a temperature below 300°.

26. A process according to any of the previous claims 2 to 25, wherein in step (i) and step (vii) the same reactor type is used, differentiated by the presence of a cooling device for step (i) and a heating device for step (vii), respectively.

27. A process according to any of the previous claims 2 to 26, wherein step (i) and step (vii) are operated with the same reactor type with the following differences:
(a) a methanol evaporating, compressing and heating unit,
(b) a heating device for the main temperature regulating cycle,
(c) reversal of the post-reactor recyclization unit so that the gaseous phase (syngas) is sent downstream and the liquid stream (methanol) is recycled back into the reactor.

28. A process according to any of the previous claims 2 to 24 and 26 to 27, wherein 1 wt.-% to 90 wt.-% water based on the amount by weight of methanol, preferably 10 wt.-% to 85 wt.-%, more preferably 20 wt.-% to 70 wt.-%, most preferably 20 wt.-% to 50 wt.-% water in addition to methanol is added in step (vii).

29. A process according to any of the previous claims 2 to 27, wherein the methanol decomposition of step (vii) is performed in a methanol decomposer with dry and CO₂ free methanol,
wherein a hydrogen enriched stream, preferably comprising at least 90 mol-% of H₂, is separated from the syngas stream obtained from step (vii), and wherein the remaining CO enriched stream, preferably comprising at least 10 mol-% of CO, is fed into a shaft furnace for the reduction of iron oxides to iron of step (x).

30. The process according to the previous claims 2 to 24 and 26 to 28, wherein
the catalyst used in the thermal catalytic decomposition of methanol in step (vii) is a ZnO-containing catalyst, preferably containing ZnO/Al₂O₃, more preferably containing Cu/ZnO/Al₂O₃, and
an amount of water is added to the methanol subjected to decomposition in step (vii), preferably 1 wt.-% to 90 wt.-% water based on the amount by weight of methanol, preferably 10 wt.-% to 85 wt.-%, more preferably 20 wt.-% to 70 wt.-%, most preferably 20 wt.-% to 50 wt.-% water in addition to methanol is added.

31. The process according to any of the previous claims 2 to 30, wherein step (vii) is performed in a shaft furnace upon vapourizing methanol and feeding it hot into the shaft furnace, followed by the reduction of iron oxides to iron of step (x).

32. The process according to the previous claim 31, wherein step (vii) and step (x) are performed at temperatures between 500° C. and 950° C., preferably between 550° C. and 850° C., more preferably between 550° C. and 700° C. and most preferably between 550° C. and 650° C.
